(19) United States
(12) Patent Application Publication
(10) Pub. No.: US 2013/0251101 A1
(43) Pub. Date: Sep. 26, 2013

(54) X-RAY CT APPARATUS AND METHOD FOR CONTROLLING THE SAME

(71) Applicants: KABUSHIKI KAISHA TOSHIBA, Tokyo (JP); TOSHIBA MEDICAL SYSTEMS CORPORATION, Otawara-shi (JP)

(72) Inventors: Hideo SAITO, Nasushiobara-shi (JP); Jyunichi KIKUHARA, Otawara-shi (JP); Yuki KATO, Otawara-shi (JP)

(73) Assignees: Toshiba Medical Systems Corporation, Otawara-shi (JP); Kabushiki Kaisha Toshiba, Tokyo (JP)

(21) Appl. No.: 13/848,363

(22) Filed: Mar. 21, 2013

(30) Foreign Application Priority Data
Mar. 21, 2012 (JP) 2012-063176

(51) Int. Cl. A61B 6/00; A61B 6/03

(52) U.S. Cl. CPC A61B 6/547; A61B 6/032
USPC 378/20

(57) ABSTRACT

An X-ray CT apparatus according to an embodiment includes: a table on which an examinee lies down; an X-ray irradiator including an X-ray tube emitting X-rays to the examinee on the table and a diaphragm blocking the X-rays and capable of opening/closing; an X-ray detector detecting X-rays transmitted through the examinee on the table after being emitted by the X-ray irradiator and passing through the diaphragm; a rotating gantry supporting the X-ray irradiator and detector; a rotation drive unit rotating the rotating gantry about a body axis of the examinee on the table; a movement drive unit moving the table in a direction of the body axis of the examinee on the table; a positional information acquirer acquiring positional information on the moving table; and a controller controlling opening and closing operations of the diaphragm of the X-ray irradiator based on the positional information on the table.

### CROSS-REFERENCE TO THE RELATED APPLICATION

This application is based on and claims the benefit of priority from Japanese Patent Applications No. 2012-63176, filed on Mar. 21, 2012; the entire contents of which are incorporated herein by reference.

### FIELD

Embodiments described herein relate generally to an X-ray CT apparatus and a method for controlling the X-ray CT apparatus.

### BACKGROUND

An X-ray CT apparatus (X-ray computed tomography imaging apparatus) irradiates an examinee such as a patient with X-rays and detects X-rays transmitted through the examinee. A data collecting device of the X-ray CT apparatus then collects X-ray transmission data which is based on the amount of X-rays detected. Thereafter, the X-ray CT apparatus performs reconstruction processing on the X-ray transmission data, and generates a slice image (a tomographic image) of the examinee. An example of such an X-ray CT apparatus which has been developed is configured to image an examinee using an X-ray irradiator and an X-ray detector located opposite each other with the examinee on a table of a bed in between, while rotating them about the body axis of the examinee.

In order to change an X-ray radiation field, the X-ray CT apparatus includes an X-ray diaphragm, such as a collimator, below an X-ray tube of the X-ray irradiator. For example, this X-ray diaphragm includes components such as a pair of blades (slit plates) configured to block X-rays and a movement mechanism configured to move the blades in directions away from and toward each other. The X-ray diaphragm changes the X-radiation field by adjusting the width of an opening which is formed by the pair of blades and through which X-rays transmit.

Such an X-ray CT apparatus performs imaging by helical scan. In the helical scan, the X-ray irradiator and the X-ray detector are rotated about the body axis of an examinee lying down on a table while the table is moved at a constant speed along the body axis of the examinee, i.e., either in a direction from the feet to the head or in a direction from the head to the feet.

In this imaging, provided that the moving speed of the table is constant (within an allowable range), timings for starting an opening operation and a closing operation of the X-ray diaphragm are controlled based on a time period almost proportional to the number of views, namely, the number of rotations (rotational speed) of the X-ray irradiator and the X-ray detector about the body axis of the examinee (a time period converted from the number of views). Note that the opening operation is to move the pair of blades away from each other, while the closing operation is to move them toward each other.

However, in variable Helical Pitch (vHP) scan and shuttle helical scan, the moving speed of the table is not constant like in the helical scan described above, but the table moves at a variable speed or shuttles. For this reason, it is difficult to perform control as intended according to the moving speed of the table. As a result, the opening and closing operations of the X-ray diaphragm might be performed too early or too late.

For example, assume that only a certain site of an examinee is to be imaged. If the opening operation starts early, the examinee is exposed to radiation more than necessary, and if the opening operation starts late, the amount of X-ray transmission data obtained is insufficient, which makes the resultant image unreliable (incomplete). Similarly, if the closing operation starts early, the amount of X-ray transmission data obtained is insufficient, which makes the resultant image unreliable (incomplete), and if the closing operation starts late, the examinee is exposed to radiation more than necessary.

### BRIEF DESCRIPTION OF THE DRAWINGS

FIG. 1 is a diagram schematically showing the configuration of an X-ray CT apparatus according to an embodiment.

FIG. 2 is a diagram schematically showing the configurations of an X-ray diaphragm and a controller of the X-ray CT apparatus according to the embodiment.

FIG. 3 is a diagram schematically showing the configuration of the controller, together with a bed of the X-ray CT apparatus and part of an imaging device.

FIG. 4 is a flowchart showing a flow of imaging processing performed by the X-ray CT apparatus according to the embodiment.

### DETAILED DESCRIPTION

According to one embodiment, an X-ray CT apparatus comprises: a table on which an examinee lies down; an X-ray irradiator including an X-ray tube configured to emit X-rays to the examinee on the table and a diaphragm configured to block the X-rays and capable of opening and closing; an X-ray detector configured to detect X-rays transmitted through the examinee on the table after being emitted by the X-ray irradiator and passing through the diaphragm; a rotating gantry configured to support the X-ray irradiator and the X-ray detector; a rotation drive unit configured to rotate the rotating gantry about a body axis of the examinee on the table; a movement drive unit configured to move any one of the table and the rotating gantry in a direction of the body axis of the examinee on the table; a positional information acquirer configured to acquire positional information on a mobile object which is any one of the table and the rotating gantry moved by the movement drive unit; and a controller configured to control opening and closing operations of the diaphragm of the X-ray irradiator based on the positional information acquired by the positional information acquirer.

According to another embodiment, provided is a method for controlling an X-ray CT apparatus including a table on which an examinee lies down, an X-ray irradiator having an X-ray tube configured to emit X-rays to the examinee on the table and a diaphragm configured to block the X-rays and capable of opening and closing, an X-ray detector configured to detect X-rays which are emitted by the X-ray irradiator, pass through the diaphragm, and then transmit through the examinee on the table, a rotating gantry configured to support the X-ray irradiator and the X-ray detector, a rotation drive unit configured to rotate the rotating gantry about a body axis of the examinee on the table, and a movement drive unit configured to move any one of the table and the rotating gantry in a direction of the body axis of the examinee on the table. The method comprises the steps of: acquiring, by a positional information acquirer, positional information on a mobile object which is any one of the table and the rotating gantry moved by the movement drive unit; and controlling, by a controller, opening and closing operations of the diaphragm of the X-ray irradiator based on the positional information acquired by the positional information acquirer.

An embodiment is described with reference to the drawings.

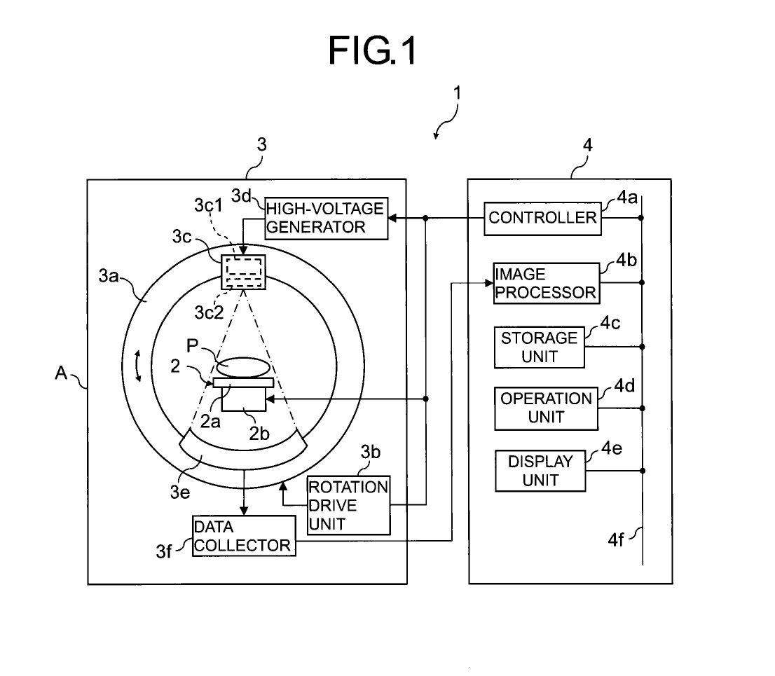

As shown in FIG. 1, an X-ray CT apparatus (X-ray computed tomography imaging apparatus) 1 according to this embodiment includes a bed 2 on which an examinee P, such as a patient, lies down, an imaging device 3 configured to image the examinee P on the bed 2, and a control device 4 configured to control the bed 2 and the imaging device 3.

The bed 2 includes a rectangular table 2a on which the examinee P is placed and a movement drive unit 2b configured to support the table 2a and move the table 2a in horizontal directions and vertical directions (up and down directions). The movement drive unit 2b has a movement mechanism for moving the table 2a, a drive source for supplying a driving power for moving the table 2a, and the like. The movement drive unit 2b of the bed 2 moves the table 2a up to a desired height and then moves the table 2a horizontally to transfer the examinee P on the table 2a to a desired position.

The imaging device 3 includes a rotator 3a provided rotatably inside a gantry A, which is a chassis, a rotation drive unit 3b configured to rotate the rotator 3a, an X-ray irradiator 3c configured to emit X-rays, a high-voltage generator 3d configured to supply the X-ray irradiator 3c with a high voltage, an X-ray detector 3e configured to detect X-rays transmitted through the examinee P on the table 2a, and a data collector 3f configured to collect the X-rays detected by the X-ray detector 3e as X-ray transmission data (X-ray amount distribution data).

The rotator 3a is a ring-shaped rotational frame (rotating gantry) configured to support components such as the X-ray irradiator 3c and the X-ray detector 3e and rotate. The rotator 3a is held by the gantry A rotatably. The X-ray irradiator 3c and the X-ray detector 3e are located on the rotator 3a at positions opposite each other so that the examinee P on the table 2a may be located in between them, and they rotate around the examinee P about the body axis of the examinee P.

The rotation drive unit 3b is located inside the gantry A of the imaging device 3. The rotation drive unit 3b drives the rotator 3a to rotate as controlled by the control device 4. For example, the rotation drive unit 3b rotates the rotator 3a in one direction at a predetermined rotation speed based on a control signal sent from the control device 4.

The X-ray irradiator 3c is fixed to the rotator 3a and includes an X-ray tube 3c1 configured to emit X-rays and an X-ray diaphragm 3c2, such as a collimator, configured to narrow the X-rays emitted by the X-ray tube 3c1. Specifically, the X-ray irradiator 3c is configured such that X-rays emitted by the X-ray tube 3c1 are narrowed by the X-ray diaphragm 3c2 so that the examinee P on the table 2a may be irradiated with a beam of X-rays having a fan beam shape with a cone angle, e.g., a pyramid shape.

The high-voltage generator 3d is located inside the gantry A of the imaging device 3. The high-voltage generator 3d is a device for generating a high voltage to be supplied to the X-ray tube 3c1 of the X-ray irradiator 3c, and is configured to step-up or rectify a voltage given by the control device 4 and supply the stepped-up or rectified voltage to the X-ray tube 3c1. To cause the X-ray tube 3c1 to generate X-rays as desired, the control device 4 controls the waveform of a voltage to give to the high-voltage generator 3d, i.e., various conditions such as the amplitude and pulse width.

The X-ray detector 3e is fixed to the rotator 3a at a position opposite the X-ray irradiator 3c. The X-ray detector 3e converts X-rays transmitted through the examinee P on the table 2a into electric signals and sends them to the data collector 3f. As the X-ray detector 3e, a multi-layered, multichannel X-ray detector can be used. The multilayered, multichannel X-ray detector is configured with X-ray detection elements configured to detect X-rays and arranged in lattice. Specifically, a channel is formed by multiple (e.g., several hundreds to several thousands of) X-ray detection elements arranged in a channel direction (i.e., a direction about the body axis of the examinee P), and multiple (e.g., 16 or 64) rows of such a channel are arranged in a slice direction (i.e., in a direction of the body axis of the examinee P).

The data collector 3f is located inside the gantry A of the imaging device 3, and configured to collect the electrical signals sent from the X-ray detector 3c as X-ray transmission data (X-ray amount distribution data), and send this X-ray transmission data to the control device 4.

The control device 4 includes a controller 4a configured to control each unit, an image processor 4b configured to perform various kinds of image processing on the X-ray transmission data, a storage unit 4c configured to store various programs, various kinds of data, and the like, an operation unit 4d configured to receive an operation inputted by the user, and a display unit 4e configured to display images. The controller 4a, the image processor 4b, the storage unit 4c, the operation unit 4d, and the display unit 4e are electrically connected to each other via a bus line 4f.

Based on the various programs and data stored in the storage unit 4c, the controller 4a controls units such as the movement drive unit 2b of the bed 2 and the rotation drive unit 3b and the high-voltage generator 3d of the imaging device 3. In addition, the controller 4a controls the diaphragm 3c2 of the X-ray irradiator 3c, and also controls display of various images, such as slice images (tomographic images) and scanograms (positioning images), on the display unit 4e. For example, a central processing unit (CPU) or the like can be used as the controller 4a.

The image processor 4b performs various types of image processing, such as preprocessing for obtaining projection data from the X-ray transmission data sent from the data collector 3f, image reconstruction processing for performing image reconstruction on the projection data, and scanogram generation processing for generating scanograms. For example, an array processor or the like can be used as this image processor 4b.

The storage unit 4c is a storage device configured to store various programs, various kinds of data, and the like. Examples of the various kinds of data include slice images and scanograms. For example, a read-only memory (ROM), a random access memory (RAM), a hard disk (magnetic disk device), a flash memory (semiconductor disk device), or the like can be used as the storage unit 4c.

The operation unit 4d is an input unit configured to receive various operations inputted on the input unit 4d by a user, such as instructing imaging, displaying an image, switching between images, and making various settings. For example, input devices such as a keyboard, a mouse, and a control lever can be used as the operation unit 4d.

The display unit 4e is a display device configured to display various types of images, such as an X-ray image and a scanogram of the examinee P and an operation screen. For example, a liquid crystal display, a CRT-based display, or the like can be used as the display unit 4e.

In the X-ray CT apparatus 1 configured as above, in response to an operation inputted on the operation unit 4d by the user, the movement drive unit 2b inserts the table 2a on which the examinee P is placed into the frame-shaped rotator 3a, and moves the examinee P on the table 2a in its body axis direction (e.g., in a direction from the feet to the head). In the X-ray CT apparatus 1, while the table 2a is moved, the rotation drive unit 3b rotates the rotator 3a so that the X-ray irradiator 3c and the X-ray detector 3e rotate about the body axis of the examinee P on the table 2a. During this rotation of the rotary 3a, the examinee P is imaged by the X-ray irradiator 3c irradiating the examinee P with X-rays and by the X-ray detector 3e detecting X-rays transmitted through the examinee P (i.e., this imaging is performed by the helical scan). In this event, in the X-ray CT apparatus 1, the data collector 3f collects electrical signals from the X-ray detector 3e as projection data, and the image processor 4b processes the projection data and saves the processed X-ray image (slice image) in the storage unit 4c. The X-ray image is displayed on the display unit 4e.

There are three imaging modes herein: a helical scan mode, a variable helical pitch scan mode, and a shuttle helical scan mode. In the helical scan mode, the examinee P is imaged while the table 2a is moved at a constant speed (within an allowable speed range) in one direction along the body axis of the examinee P (e.g., from the feet to the head). In the variable helical pitch scan mode, the moving speed of the table 2a is changed for, for example, a site targeted for the imaging. In the shuttle helical scan mode, the examinee P is imaged while the table 2a is moved with its moving direction being changed alternately between two directions along the body axis of the examinee P (e.g., in a direction from the feet to the head and in a direction from the head to the feet). The X-ray CT apparatus 1 is thus capable of performing X-ray imaging by various imaging modes.

Next, the X-ray diaphragm 3c2 described earlier is described in detail.

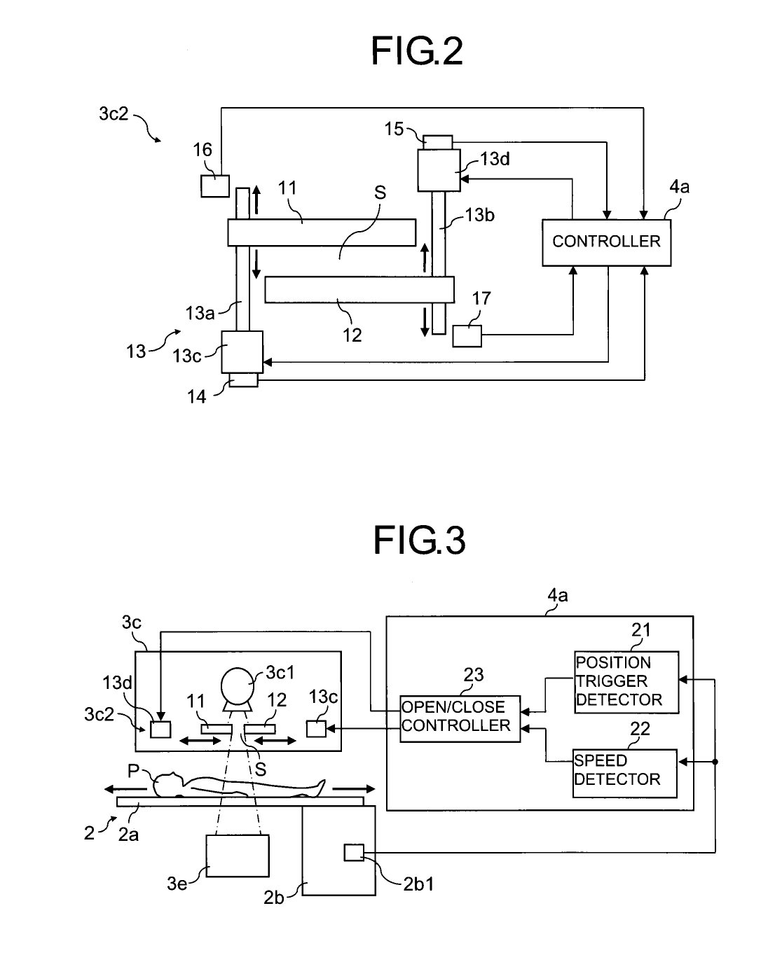

As shown in FIG. 2, the X-ray diaphragm 3c2 includes a pair of blades 11 and 12 configured to narrow a beam of X-rays by blocking them, a blade moving mechanism 13 configured to move the blades 11 and 12 in such directions as to change the opening width of a slit S which is an opening for narrowing the beam (in directions of the body axis of the examinee P), two encoders 14 and 15 configured to check the positions of the blades 11 and 12, respectively, and original-position sensors 16 and 17 configured to determine the original positions of the blades 11 and 12, respectively.

The blade moving mechanism 13 includes two shafts 13a and 13b configured to respectively guide the blades 11 and 12 in such directions as to change the opening width of the slit S, and two motors 13c and 13d which are drive sources for moving the blades 11 and 12, respectively. For example, pulse motors can be used as the motors 13c and 13d. The motors 13c and 13d are electrically connected to the controller 4a, and are driven as controlled by the controller 4a. As the blade moving mechanism 13, a feed-screw mechanism can for example be used.

The encoders 14 and 15, which are electrically connected to the controller 4a, detect the amounts of movement of the blades 11 and 12, respectively, and input the detected amounts to the controller 4a. Upon detection of the blades 11 and 12, the original-position sensors 16 and 17, which are electrically connected to the controller 4a, input detection signals to the controller 4a. Thus, the controller 4a can know the zero points of the encoders 14 and 15.

In the X-ray diaphragm 3c2 thus configured, first, the motors 13c and 13d are driven at a predetermined timing, such as before imaging, to move the pair of blades 11 and 12 to the positions of the original-position sensors 16 and 17, respectively, so that the controller 4a can know the zero points of the encoders 14 and 15. Then, as controlled by the controller 4a, the X-ray diaphragm 3c2 performs an opening operation for moving the blades 11 and 12 to opening positions where the opening width of the slit S is at a preset value or a closing operation for moving the blades 11 and 12 to closing positions where the opening width is zero. The X-ray radiation field for the X-ray detector 3e can be adjusted by changing the above preset value of the opening width.

Note that various types of X-ray diaphragm can be used as the X-ray diaphragm 3c2, besides the above described X-ray diaphragm configured to change the size of the opening formed by the blades 11 and 12, which are X-ray blocking plates made of lead or the like, by moving the blades 11 and 12 in directions away from and toward each other.

Next, the controller 4a mentioned above is described in detail.

As shown in FIG. 3, the controller 4a includes a position trigger detector 21 configured to detect a timing for starting the opening or closing operation of the X-ray diaphragm 3c2 based on positional information on the moving table 2a, a speed detector 22 configured to detect a moving speed of the table 2a based on the positional information on the moving table 2a, and an open/close controller 23 configured to control driving of the motors 13d and 13c of the X-ray diaphragm 3c2.

Note that the movement drive unit 2b of the bed 2 has a positional information acquirer 2b1 configured to acquire the positional information on the table 2a which is a mobile object and to output the acquired positional information to the position trigger detector 21 and the speed detector 22. An encoder, for example, can be used as the positional information acquirer 2b1. The encoder is attached to, for example, the motor of the movement drive unit 2b.

The position trigger detector 21 receives the positional information on the table 2a from the positional information acquirer 2b1 of the bed 2, and then detects a timing for starting the opening or closing operation based on a comparison between the acquired positional information on the table 2a and a start position for the opening or closing operation of the X-ray diaphragm 3c2. Then, the position trigger detector 21 outputs a detection signal notifying of the start timing to the open/close controller 23. For example, a comparator can be used as the position trigger detector 21.

The start position for the opening or closing operation of the X-ray diaphragm 3c2 described above is set in advance according for example to an imaged area, such as which part of the examinee to image (e.g., an organ or a site), determined at the time of imaging planning or the like.

The speed detector 22 receives the positional information from the positional information acquirer 2b1 of the bed 2, and then derives the moving speed of the table 2a from the positional information on the table 2a acquired. More specifically, the speed detector 22 calculates the amount of movement of the table 2a per unit time from the positional information on the moving table 2a acquired successively and thus obtains the moving speed of the table 2a. Then, the speed detector 22 outputs data on the moving speed of the table 2a to the open/close controller 23.

Based on the detection signal (trigger signal) outputted from the position trigger detector 21 and the data on the moving speed of the table 2a outputted from the speed detector 22, the open/close controller 23 controls the opening or closing operation of the X-ray diaphragm 3c2 (an operation for moving the pair of blades 11 and 12 in the directions of the body axis of the examinee P), in other words, controls the motors 13d and 13c of the X-ray diaphragm 3c2.

For example, upon receipt of the detection signal for the opening operation, the open/close controller 23 starts the opening operation of the X-ray diaphragm 3c2, i.e., starts rotating the motors 13d and 13c in such directions as to move the pair of blades 11 and 12 away from each other. Further, the open/close controller 23 controls the motors 13d and 13c with an electronic gear multiplied by a multiplication/division rate obtained based to the data on the moving speed of the table 2a.

Upon receipt of the detection signal for the closing operation, the open/close controller 23 starts the closing operation of the X-ray diaphragm 3c2, i.e., starts rotating the motors 13d and 13c in such directions as to move the pair of blades 11 and 12 toward each other. As is similar to the case of the opening operation, the open/close controller 23 controls the motors 13d and 13c with an electronic gear multiplied by a multiplication/division rate obtained based to the data on the moving speed of the table 2a.

Note that the position trigger detector 21, the speed detector 22, and the open/close controller 23 may be configured by hardware such as electric circuits, or may be configured by software such as programs executing their functions, or may be configured by a combination of both.

Next, a description is given of imaging processing performed by the X-ray CT apparatus 1. Note that the variable helical pitch scan mode and the shuttle helical scan mode are set as the imaging modes, and imaging is performed by either the variable helical pitch scan or the shuttle helical scan.

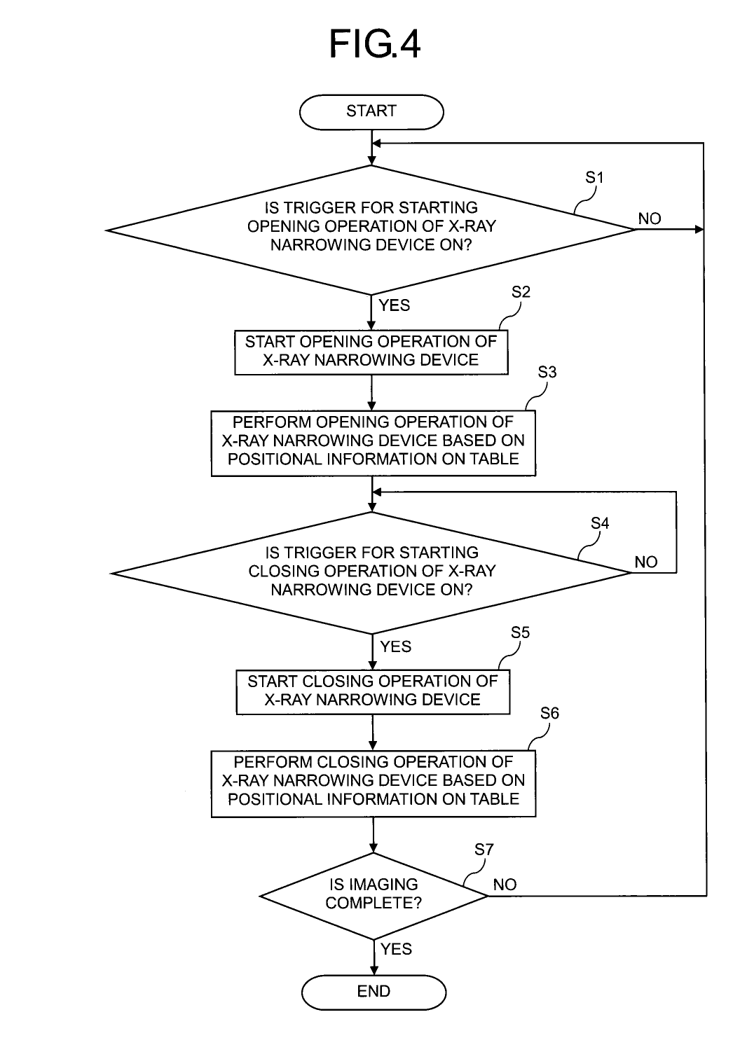

As shown in FIG. 4, first, it is determined based on positional information on the table 2a whether or not a trigger for starting the opening operation of the X-ray diaphragm 3c2 is turned on (Step S1). This step is repeated until the trigger for starting the opening operation is turned on (NO in Step S1).

Specifically, in Step S1, the trigger for starting the opening operation is determined as being turned on when the open/close controller 23 receives a detection signal (trigger signal) for the opening operation from the position trigger detector 21.

When it is determined in Step S1 that the trigger for starting the opening operation of the X-ray diaphragm 3c2 is turned on (YES in Step S1), the opening operation of the X-ray diaphragm 3c2 is started (Step S2), and the opening operation of the X-ray diaphragm 3c2 is performed based on the positional information on the table 2a (Step S3).

Specifically, in Step S2, the opening operation of the X-ray diaphragm 3c2 is started, i.e., an operation for rotating the motors 13d and 13c by a predetermined amount in such directions as to move the pair of blades 11 and 12 away from each other is started. Further, in Step S3, the motors 13d and 13c (speed of the opening operation) are controlled by an electronic gear multiplied by a multiplication/division rate obtained based on a moving speed of the table 2a which is derived from the positional information on the table 2a.

Next, after the processing in Step S3, it is determined based on the positional information on the table 2a whether or not a trigger for starting the closing operation of the X-ray diaphragm 3c2 is turned on (Step S4). This step is repeated until the trigger for starting the closing operation is turned on (NO in Step S4).

Specifically, in Step S4, the trigger for starting the closing operation is determined as being turned on when the open/close controller 23 receives the detection signal (trigger signal) for the closing operation from the position trigger detector 21.

When it is determined in Step S4 that the trigger for starting the closing operation of the X-ray diaphragm 3c2 is turned on (YES in Step S4), the closing operation of the X-ray diaphragm 3c2 is started (Step S5), and the closing operation of the X-ray diaphragm 3c2 is performed based on the positional information on the table 2a (Step S6).

Specifically, in Step S5, the closing operation of the X-ray diaphragm 3c2 is started, i.e., an operation for rotating the motors 13d and 13c by a predetermined amount in such directions as to move the pair of blades 11 and 12 toward each other is started. Further, in Step S6, as is similar to the case of the opening operation, the motors 13d and 13c (speed of the closing operation) are controlled by an electronic gear multiplied by a multiplication/division rate obtained based on a moving speed of the table 2a which is derived from the positional information on the table 2a.

After the processing in Step S6, it is determined whether imaging is completed or not (Step S7). The processing proceeds back to Step S1 to repeat the steps from Step S1 when the imaging is not completed (NO in Step S7), and the processing ends when the imaging is completed (YES in Step S7).

In Step S7, the determination of whether imaging is completed or not is made by, for example, judging whether the table 2a is at a predetermined imaging complete position or not. As an example, when the imaging mode is the variable helical pitch scan mode or the shuttle helical scan mode, the determination in Step S7 is made based on a judgment on whether or not the table 2a is at the predetermined imaging completion position reached after the variable helical pitch scan or the shuttling helical scan is completed. Then, it is determined that the imaging is completed when it is judged that the table 2a is at the predetermined imaging completion position.

According to such imaging processing, the opening operation of the X-ray diaphragm 3c2 is started at a start timing which is based on a comparison between the positional information on the table 2a and the start position for the opening operation of the X-ray diaphragm 3c2. Similarly, the closing operation of the X-ray diaphragm 3c2 is started at a start timing which is based on a comparison between the positional information on the table 2a and the start position for the closing operation of the X-ray diaphragm 3c2. In addition, the opening operation and the closing operation of the X-ray diaphragm 3c2 are controlled according to the moving speed of the table 2a derived from the positional information on the table 2a, and consequently controlled in such a manner as to follow the variable moving speed of the table 2a. In this way, the opening and closing operations can be controlled accurately independent of the variable (inconstant) moving speed of the table 2a, i.e., independent of the inconstancy in the trigger for starting the opening and closing operations of the X-ray diaphragm 3c2 due to the number of views. Consequently, the timings for starting the opening operation and the closing operation are no longer too early or too late, so as to prevent unnecessary exposure to radiation and to allow reliable (complete) imaging with necessary amount of X-ray transmission data.

As described above, according to the embodiment, the positional information acquirer 2b1 acquires position information on the mobile table 2a, and the controller 4a controls the opening and closing operations of the X-ray diaphragm 3c2 of the X-ray irradiator 3c based on the positional information on the table 2a acquired. Thus, being controlled based on the positional information on the table 2a, the opening and closing operations of the X-ray diaphragm 3c2 can be controlled accurately not dependent on the change (inconstancy) in the moving speed of the table 2a. As a result, reduction in exposure to radiation as well as reliable imaging can be achieved.

In particular, when the position trigger detector 21 detects a timing for staring the opening or closing operation of the X-ray diaphragm 3c2, the corresponding one of the opening operation and the closing operation is started, and the speed of the opening or closing operation of the X-ray diaphragm 3c2 is controlled based on the moving speed of the table 2a derived by the speed detector 22. Thus, the accurate control of the opening/closing operation can be reliably performed, and therefore the exposure to radiation and the reliable imaging can be achieved even more surely.

Although the controller 4a uses the positional information on the mobile table 2a during the imaging in the embodiment described above, the present invention is not limited to this. For example, if the X-ray CT apparatus 1 is of a type where the gantry A, not the table 2a, is moved by a moving drive unit (including, for example, a rail mechanism, a drive source, a positional information acquirer, and the like) during the imaging, the gantry A, which includes the X-ray irradiator 3c, the X-ray detector 3e, the rotator 3a, and the like, is a mobile object. In such a case, the controller 4a uses positional information on the gantry A as the positional information on the mobile object.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An X-ray CT apparatus comprising:
a table on which an examinee lies down;
an X-ray irradiator including an X-ray tube configured to emit X-rays to the examinee on the table and a diaphragm configured to block the X-rays and capable of opening and closing;
an X-ray detector configured to detect X-rays transmitted through the examinee on the table after being emitted by the X-ray irradiator and passing through the diaphragm;
a rotating gantry configured to support the X-ray irradiator and the X-ray detector;
a rotation drive unit configured to rotate the rotating gantry about a body axis of the examinee on the table;
a movement drive unit configured to move any one of the table and the rotating gantry in a direction of the body axis of the examinee on the table;
a positional information acquirer configured to acquire positional information on a mobile object which is any one of the table and the rotating gantry moved by the movement drive unit; and
a controller configured to control opening and closing operations of the diaphragm of the X-ray irradiator based on the positional information acquired by the positional information acquirer.

2. The X-ray CT apparatus according to claim 1, wherein
the controller includes:
a position trigger detector configured to compare the positional information on the mobile object with a start position for the opening operation of the diaphragm of the X-ray irradiator and thereby detect a timing for starting the opening operation of the diaphragm of the X-ray irradiator;
a speed detector configured to derive a moving speed of the mobile object from the positional information on the mobile object; and
an open/close controller configured to start the opening operation of the diaphragm of the X-ray irradiator when the position trigger detector detects the timing for staring the opening operation, and control a speed of the opening operation of the diaphragm of the X-ray irradiator according to the moving speed of the mobile object derived by the speed detector.

3. The X-ray CT apparatus according to claim 1, wherein
the controller includes:
a position trigger detector configured to compare the positional information on the mobile object with a start position for the closing operation of the diaphragm of the X-ray irradiator and thereby detect a timing for starting the closing operation of the diaphragm of the X-ray irradiator;
a speed detector configured to derive a moving speed of the mobile object from the positional information on the mobile object; and
an open/close controller configured to start the closing operation of the diaphragm of the X-ray irradiator when the position trigger detector detects the timing for staring the closing operation, and control a speed of the closing operation of the diaphragm of the X-ray irradiator according to the moving speed of the mobile object derived by the speed detector.

4. The X-ray CT apparatus according to claim 2, wherein
the controller includes:
a position trigger detector configured to compare the positional information on the mobile object with a start position for the closing operation of the diaphragm of the X-ray irradiator and thereby detect a timing for starting the closing operation of the diaphragm of the X-ray irradiator;
a speed detector configured to derive a moving speed of the mobile object from the positional information on the mobile object; and
an open/close controller configured to start the closing operation of the diaphragm of the X-ray irradiator when the position trigger detector detects the timing for staring the closing operation, and control a speed of the closing operation of the diaphragm of the X-ray irradiator according to the moving speed of the mobile object derived by the speed detector.

5. The X-ray CT apparatus according to claim 1, further comprising a gantry configured to hold the rotating gantry such that the rotating gantry is rotatable, wherein
the movement drive unit moves the gantry in the direction of the body axis of the examinee on the table, and
the positional information acquirer acquires positional information on the gantry as the positional information on the rotating gantry moved by the movement drive unit.

6. The X-ray CT apparatus according to claim 2, further comprising a gantry configured to hold the rotating gantry such that the rotating gantry is rotatable, wherein
the movement drive unit moves the gantry in the direction of the body axis of the examinee on the table, and
the positional information acquirer acquires positional information on the gantry as the positional information on the rotating gantry moved by the movement drive unit.

7. The X-ray CT apparatus according to claim 3, further comprising a gantry configured to hold the rotating gantry such that the rotating gantry is rotatable, wherein
the movement drive unit moves the gantry in the direction of the body axis of the examinee on the table, and
the positional information acquirer acquires positional information on the gantry as the positional information on the rotating gantry moved by the movement drive unit.

8. The X-ray CT apparatus according to claim 4, further comprising a gantry configured to hold the rotating gantry such that the rotating gantry is rotatable, wherein
the movement drive unit moves the gantry in the direction of the body axis of the examinee on the table, and
the positional information acquirer acquires positional information on the gantry as the positional information on the rotating gantry moved by the movement drive unit.

9. A method for controlling an X-ray CT apparatus including a table on which an examinee lies down, an X-ray irradiator having an X-ray tube configured to emit X-rays to the examinee on the table and a diaphragm configured to block the X-rays and capable of opening and closing, an X-ray detector configured to detect X-rays transmitted through the examinee on the table after being emitted by the X-ray irradiator and passing through the diaphragm, a rotating gantry configured to support the X-ray irradiator and the X-ray detector, a rotation drive unit configured to rotate the rotating gantry about a body axis of the examinee on the table, and a movement drive unit configured to move any one of the table and the rotating gantry in a direction of the body axis of the examinee on the table, the method comprising the steps of:
acquiring, by a positional information acquirer, positional information on a mobile object which is any one of the table and the rotating gantry moved by the movement drive unit; and
controlling, by a controller, opening and closing operations of the diaphragm of the X-ray irradiator based on the positional information acquired by the positional information acquirer.

10. The method for controlling an X-ray CT apparatus according to claim 9, wherein
in the controlling step,
the opening operation of the diaphragm of the X-ray irradiator is started when a timing for starting the opening operation of the diaphragm of the X-ray irradiator is detected by a comparison between the positional information on the mobile object and a start position for the opening operation of the diaphragm of the X-ray irradiator, and
a speed of the opening operation of the diaphragm of the X-ray irradiator is controlled according to a moving speed of the mobile object derived from the positional information on the mobile object.

11. The method for controlling an X-ray CT apparatus according to claim 9, wherein
in the controlling step,
the closing operation of the diaphragm of the X-ray irradiator is started when a timing for starting the closing operation of the diaphragm of the X-ray irradiator is detected by a comparison between the positional information on the mobile object and a start position for the closing operation of the diaphragm of the X-ray irradiator, and
a speed of the closing operation of the diaphragm of the X-ray irradiator is controlled according to a moving speed of the mobile object derived from the positional information on the mobile object.

12. The method for controlling an X-ray CT apparatus according to claim 10, wherein
in the controlling step,
the closing operation of the diaphragm of the X-ray irradiator is started when a timing for starting the closing operation of the diaphragm of the X-ray irradiator is detected by a comparison between the positional information on the mobile object and a start position for the closing operation of the diaphragm of the X-ray irradiator, and
a speed of the closing operation of the diaphragm of the X-ray irradiator is controlled according to a moving speed of the mobile object derived from the positional information on the mobile object.

13. The method for controlling an X-ray CT apparatus according to claim 9, wherein
the X-ray CT apparatus further includes a gantry configured to hold the rotating gantry such that the rotating gantry is rotatable,
the movement drive unit moves the gantry in the direction of the body axis of the examinee on the table, and
in the acquiring step, positional information on the gantry is acquired as the positional information on the rotating gantry moved by the movement drive unit.

14. The method for controlling an X-ray CT apparatus according to claim 10, wherein
the X-ray CT apparatus further includes a gantry configured to hold the rotating gantry such that the rotating gantry is rotatable,
the movement drive unit moves the gantry in the direction of the body axis of the examinee on the table, and
in the acquiring step, positional information on the gantry is acquired as the positional information on the rotating gantry moved by the movement drive unit.

15. The method for controlling an X-ray CT apparatus according to claim 11, wherein
the X-ray CT apparatus further includes a gantry configured to hold the rotating gantry such that the rotating gantry is rotatable,
the movement drive unit moves the gantry in the direction of the body axis of the examinee on the table, and
in the acquiring step, positional information on the gantry is acquired as the positional information on the rotating gantry moved by the movement drive unit.

16. The method for controlling an X-ray CT apparatus according to claim 12, wherein
the X-ray CT apparatus further includes a gantry configured to hold the rotating gantry such that the rotating gantry is rotatable,
the movement drive unit moves the gantry in the direction of the body axis of the examinee on the table, and
in the acquiring step, positional information on the gantry is acquired as the positional information on the rotating gantry moved by the movement drive unit.
